SEQUENCE LISTING (72 sequences)

SEQ ID NO: 1 - protein, 37 aa, Homo Sapiens
ASYQVGQDLTVMAALGLGFLTSAFRRHSWSSVAFNLF

SEQ ID NO: 2 - protein, 15 aa, Homo Sapiens
ASYQVGQDLTVMAAL

SEQ ID NO: 3 - protein, 15 aa, Homo Sapiens
SYQVGQDLTVMAALG

SEQ ID NO: 4 - protein, 15 aa, Homo Sapiens
YQVGQDLTVMAALGL

SEQ ID NO: 5 - protein, 15 aa, Homo Sapiens
QVGQDLTVMAALGLG

SEQ ID NO: 6 - protein, 15 aa, Homo Sapiens
VGQDLTVMAALGLGF

SEQ ID NO: 7 - protein, 15 aa, Homo Sapiens
GQDLTVMAALGLGFL

SEQ ID NO: 8 - protein, 15 aa, Homo Sapiens
QDLTVMAALGLGFLT

SEQ ID NO: 9 - protein, 15 aa, Homo Sapiens
DLTVMAALGLGFLTS

SEQ ID NO: 10 - protein, 15 aa, Homo Sapiens
LTVMAALGLGFLTSA

SEQ ID NO: 11 - protein, 15 aa, Homo Sapiens
TVMAALGLGFLTSAF

SEQ ID NO: 12 - protein, 15 aa, Homo Sapiens
VMAALGLGFLTSAFR

SEQ ID NO: 13 - protein, 15 aa, Homo Sapiens
MAALGLGFLTSAFRR

SEQ ID NO: 14 - protein, 15 aa, Homo Sapiens
AALGLGFLTSAFRRH

SEQ ID NO: 15 - protein, 15 aa, Homo Sapiens
ALGLGFLTSAFRRHS

SEQ ID NO: 16 - protein, 15 aa, Homo Sapiens
LGLGFLTSAFRRHSW

SEQ ID NO: 17 - protein, 15 aa, Homo Sapiens
GLGFLTSAFRRHSWS

SEQ ID NO: 18 - protein, 15 aa, Homo Sapiens
LGFLTSAFRRHSWSS

SEQ ID NO: 19 - protein, 15 aa, Homo Sapiens
GFLTSAFRRHSWSSV

SEQ ID NO: 20 - protein, 15 aa, Homo Sapiens
FLTSAFRRHSWSSVA

SEQ ID NO: 21 - protein, 15 aa, Homo Sapiens
LTSAFRRHSWSSVAF

SEQ ID NO: 22 - protein, 15 aa, Homo Sapiens
TSAFRRHSWSSVAFN

SEQ ID NO: 23 - protein, 15 aa, Homo Sapiens
SAFRRHSWSSVAFNL

SEQ ID NO: 24 - protein, 15 aa, Homo Sapiens
AFRRHSWSSVAFNLF

SEQ ID NO: 25 - protein, 29 aa, Homo Sapiens
QWAILLDGFLSQFPPGKVVITLFSIRLAT

SEQ ID NO: 26 - protein, 15 aa, Homo Sapiens
QWAILLDGFLSQFPP

SEQ ID NO: 27 - protein, 15 aa, Homo Sapiens
WAILLDGFLSQFPPG

SEQ ID NO: 28 - protein, 15 aa, Homo Sapiens
AILLDGFLSQFPPGK

SEQ ID NO: 29 - protein, 15 aa, Homo Sapiens
ILLDGFLSQFPPGKV

SEQ ID NO: 30 - protein, 15 aa, Homo Sapiens
LLDGFLSQFPPGKVV

SEQ ID NO: 31 - protein, 15 aa, Homo Sapiens
LDGFLSQFPPGKVVI

SEQ ID NO: 32 - protein, 15 aa, Homo Sapiens
DGFLSQFPPGKVVIT

SEQ ID NO: 33 - protein, 15 aa, Homo Sapiens
GFLSQFPPGKVVITL

SEQ ID NO: 34 - protein, 15 aa, Homo Sapiens
FLSQFPPGKVVITLF

SEQ ID NO: 35 - protein, 15 aa, Homo Sapiens
LSQFPPGKVVITLFS

SEQ ID NO: 36 - protein, 15 aa, Homo Sapiens
SQFPPGKVVITLFSI

SEQ ID NO: 37 - protein, 15 aa, Homo Sapiens
QFPPGKVVITLFSIR

SEQ ID NO: 38 - protein, 15 aa, Homo Sapiens
FPPGKVVITLFSIRL

SEQ ID NO: 39 - protein, 15 aa, Homo Sapiens
PPGKVVITLFSIRLA

SEQ ID NO: 40 - protein, 15 aa, Homo Sapiens
PGKVVITLFSIRLAT

SEQ ID NO: 41 - protein, 28 aa, Homo Sapiens
WRISGKWTSLNFNRMLRLLMSQYGHFPF

SEQ ID NO: 42 - protein, 15 aa, Homo Sapiens
WRISGKWTSLNFNRM

SEQ ID NO: 43 - protein, 15 aa, Homo Sapiens
RISGKWTSLNFNRML

SEQ ID NO: 44 - protein, 15 aa, Homo Sapiens
ISGKWTSLNFNRMLR

SEQ ID NO: 45 - protein, 15 aa, Homo Sapiens
SGKWTSLNFNRMLRL

SEQ ID NO: 46 - protein, 15 aa, Homo Sapiens
GKWTSLNFNRMLRLL

SEQ ID NO: 47 - protein, 15 aa, Homo Sapiens
KWTSLNFNRMLRLLM

SEQ ID NO: 48 - protein, 15 aa, Homo Sapiens
WTSLNFNRMLRLLMS

SEQ ID NO: 49 - protein, 15 aa, Homo Sapiens
TSLNFNRMLRLLMSQ

SEQ ID NO: 50 - protein, 15 aa, Homo Sapiens
SLNFNRMLRLLMSQY

SEQ ID NO: 51 - protein, 15 aa, Homo Sapiens
LNFNRMLRLLMSQYG

SEQ ID NO: 52 - protein, 15 aa, Homo Sapiens
NFNRMLRLLMSQYGH

SEQ ID NO: 53 - protein, 15 aa, Homo Sapiens
FNRMLRLLMSQYGHF

SEQ ID NO: 54 - protein, 15 aa, Homo Sapiens
NRMLRLLMSQYGHFP

SEQ ID NO: 55 - protein, 15 aa, Homo Sapiens
RMLRLLMSQYGHFPF

SEQ ID NO: 56 - protein, 15 aa, Homo Sapiens
MLRLLMSQYGHFPFF

SEQ ID NO: 57 - protein, 29 aa, Homo Sapiens
WRISGKWTSLNFNRTLRLLMSQYGHFPFF

SEQ ID NO: 58 - protein, 15 aa, Homo Sapiens
WRISGKWTSLNFNRT

SEQ ID NO: 59 - protein, 15 aa, Homo Sapiens
RISGKWTSLNFNRTL

SEQ ID NO: 60 - protein, 15 aa, Homo Sapiens
ISGKWTSLNFNRTLR

SEQ ID NO: 61 - protein, 15 aa, Homo Sapiens
SGKWTSLNFNRTLRL

SEQ ID NO: 62 - protein, 15 aa, Homo Sapiens
GKWTSLNFNRTLRLL

SEQ ID NO: 63 - protein, 15 aa, Homo Sapiens
KWTSLNFNRTLRLLM

SEQ ID NO: 64 - protein, 15 aa, Homo Sapiens
WTSLNFNRTLRLLMS

SEQ ID NO: 65 - protein, 15 aa, Homo Sapiens
TSLNFNRTLRLLMSQ

SEQ ID NO: 66 - protein, 15 aa, Homo Sapiens
SLNFNRTLRLLMSQY

SEQ ID NO: 67 - protein, 15 aa, Homo Sapiens
LNFNRTLRLLMSQYG

SEQ ID NO: 68 - protein, 15 aa, Homo Sapiens
NFNRTLRLLMSQYGH

SEQ ID NO: 69 - protein, 15 aa, Homo Sapiens
FNRTLRLLMSQYGHF

SEQ ID NO: 70 - protein, 15 aa, Homo Sapiens
NRTLRLLMSQYGHFP

SEQ ID NO: 71 - protein, 15 aa, Homo Sapiens
RTLRLLMSQYGHFPF

SEQ ID NO: 72 - protein, 15 aa, Homo Sapiens
TLRLLMSQYGHFPFF

(19) United States
(12) Patent Application Publication
(10) Pub. No.: US 2009/0170781 A1
(43) Pub. Date: Jul. 2, 2009

(54) Identification of Immunologically Effective Epitopes on the Surface of Red Blood Cells and Their Use in a Method of Inducing Tolerance Thereto

(76) Inventors: Stanislaw Joseph Urbaniak, Aberdeen (GB); Robert Norman Barker, Aberdeenshire (GB)

Correspondence Address: LICATA & TYRRELL P.C., 66 E. MAIN STREET, MARLTON, NJ, 08053, US

(21) Appl. No.: 12/096,092

(22) PCT Filed: Dec. 5, 2006
(86) PCT No.: PCT/GB2006/004539

§ 371 Date: Jul. 31, 2008

(30) Foreign Application Priority Data
Dec. 5, 2005 (GB) 0524866.1

(51) Int. Cl. A61K 38/10; C12Q 1/02

(52) U.S. Cl. 514/14; 435/29

(57) ABSTRACT

The present invention relates to a method of identifying an immunologically effective epitope, the method comprising: a) preparing an oligomer of a protein present on the surface of a red blood cell which includes a polymorphism therein and b) ascertaining whether the oligomer stimulates T-cells and a composition comprising an immunologically effective epitope for the tolerisation of a subject that has been exposed to an antithetical allele.

The present invention relates to the identification of immunodominant epitopes on red blood cells and their use in the tolerisation of individuals.

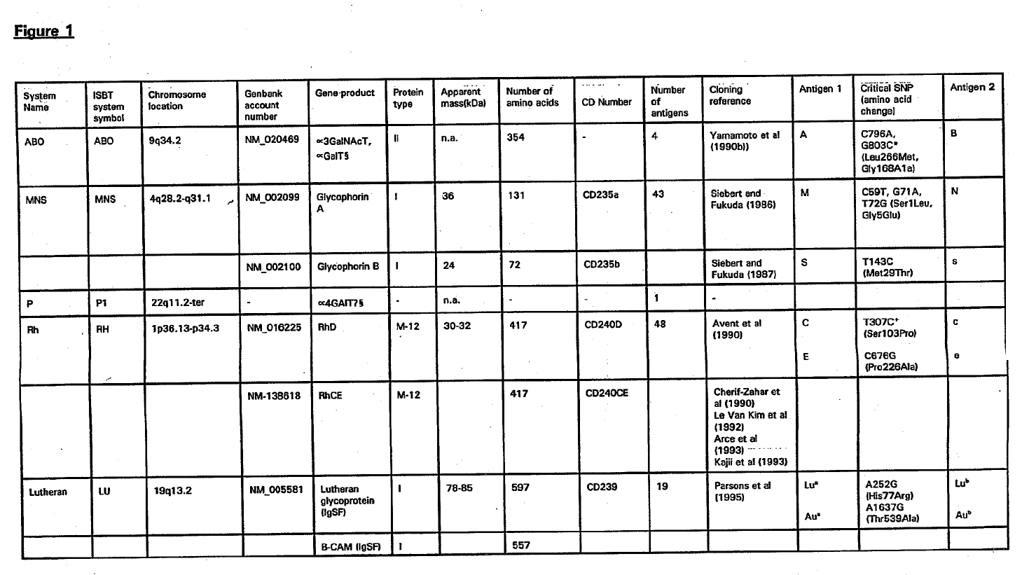

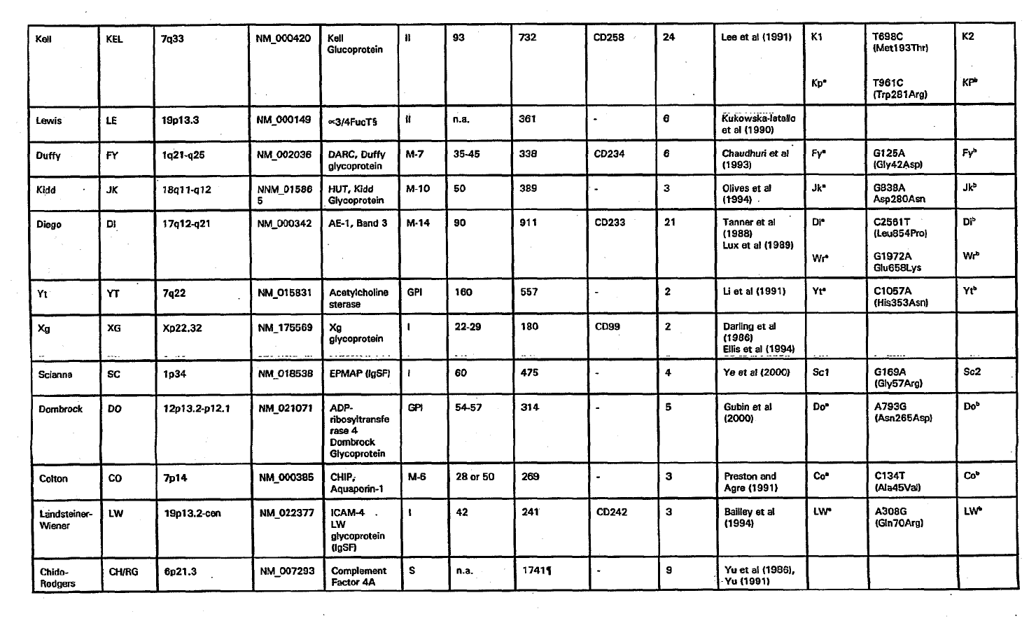

Human red cells (RBCs) express on their surface a number of antigens which are genetically determined, and differ between individuals. The genes encoding these blood group antigens have been mapped to their various chromosomes throughout the genome, and all but one of the 29 blood group genes have been characterised at the molecular level (Human Blood Groups, Daniels 2002, Blood Group Antigen Facts Book 2003, reviews Storry and Olsson 2004, Logdberg et al 2005). Apart from the ABO and Rh blood group systems, the majority of the clinically important protein blood group antigens are determined by single nucleotide polymorphisms (SNPS) at the DNA level, which are translated into critical amino acid changes, giving rise to alternative alleles at a given locus (see FIG. 1).

This complexity of the blood group system can cause problems following incompatible blood transfusion, or during pregnancy, when a women who is negative for a particular allele carries a fetus which is positive for the antithetical blood group allele. Blood for transfusion is conventionally matched only for ABO and RhD, and exposure to regular blood transfusions (e.g. as a result of sickle-cell anaemia, thalassaemia, haematological malignancies) can result in stimulation of the recipient's immune system, to produce alloantibodies to the respective allele (e.g. anti-K, anti-Fy^{a}, anti-Jk^{a}). The development of such alloantibodies, especially multiple alloantibodies, gives rise to difficulties in finding compatible blood in life-threatening situations.

In the context of pregnancy, a mother who is negative for a given allele (e.g. Kell (K1), Duffy (Fya) and Kidd (Jka)), and carries a fetus which is positive for the given allele, is at risk of being immunised by the allele positive blood cells of her own baby. This immunisation can take place during pregnancy as a result of procedures such as amniocentesis, or bleeding from the fetus to mother during antepartum haemorrhage, and also at the time of delivery. Once a mother's immune system has been exposed to the allele, she will produce antibodies against the allele, which can cross the placenta and cause haemolytic disease of the newborn in subsequent pregnancies, and such haemolytic disease can be fatal for the fetus or neonate.

There are no preventative programmes to avoid such alloimmunisations (as there is for RhD), other than the provision of compatible blood for transfusion after antibodies have been formed in the recipient, or in special cases by extended blood grouping of recipient and blood donors e.g. in thalassaemia.

An object of the present invention is to overcome the problems of the prior art.

According to the present invention there is provided a method of identifying an immunologically effective epitope, the method comprising: a) preparing an oligomer of a protein present on the surface of a red blood cell which includes a polymorphism therein and b) ascertaining whether the oligomer stimulates T-cells.

This method results in the identification of an epitope which is immunologically effective or immunodominant. Such an epitope can then be used as a tolergen in individuals that have been exposed to an antithetical allele or are at risk of exposure to such an allele.

Preferably the oligomer is between 10 and 20 nucleotides in length. More preferably the oligomer is 15 nucleotides in length. These lengths of oligomer have been found to be particularly useful as tolergens.

Conveniently a set of oligomers is prepared and/or the oligomers are overlapping. Using either or both of these steps allows the effectiveness of an array of oligomers to be tested such that the most effective oligomer can be used as a tolergen.

Preferably the polymorphism is a single nucleotide polymorphism.

Preferably the polymorphism is in the middle region of the oligomer.

Conveniently the polymorphism is unique to the protein. Use of such a polymorphism will allow the tolergen to be specific to the allele.

Preferably the protein is selected from:

ABO, MNS, P, Rh, Lutheran, Kell, Lewis, Duffy, Kidd, Diego, Yt, Xg, Scianna, Dombrock, Colton, Landsteiner-Wiener, Chido-Rodgers, Hh, Kx, Gerbich, Cromer, Knops, Indian, Ok, Raph, JMH, I, Globoside or GIL. Full identification of these proteins is given in FIG. 1.

According to a further aspect of the present invention there is provided a composition for the tolerisation of a subject that has been exposed to an antithetical allele, the composition comprising an immunologically effective epitope identified by the method set out above.

According to a yet further aspect of the present invention there is provided a composition for the tolerisation of a subject that has been exposed to an antithetical allele, the composition comprising an immunologically effective non-rhesus surface antigen protein or a peptide fragment thereof.

Exposure to antithetical alleles can result from incompatible pregnancy, incompatible blood transfusion, allogenic transplant of bone marrow, tissues and organs. The administration of such immunodominant peptides by a tolerogenic route has the potential to “switch off” the alloimmune response selectively for the relevant antigen. The provision of active peptide immunotherapy to individuals that are genetically negative for clinically important blood group antigens represents an important advance in blood transfusion medicine.

Preferably the non-rhesus surface antigen protein is selected from ABO, MNS, P, Lutheran, Kell, Lewis, Duffy, Kidd, Diego, Yt, Xg, Scianna, Dombrock, Colton, Landsteiner-Wiener, Chido-Rodgers, Hh, Kx, Gerbich, Cromer, Knops, Indian, Ok, Raph, JMH, I, Globoside or GIL.

Preferably the exposure to the antithetical allele has resulted in a condition selected from: haemolytic disease of the fetus and newborn or haemolytic transfusion reactions.

Conveniently the composition is formulated for delivery through mucosal tissue. Such a delivery method is non-invasive and in preferred embodiments the composition is not formulated for delivery through intravenous or parenteral methods.

Preferably the protein is Kell and the peptide fragment has a sequence selected from SEQ ID Nos: 42, 43, 46, 49, 58, 60 & 71.

The invention will now be described, by way of illustration only, with reference to the following examples and the accompanying figures.

FIG. 1 shows a summary of information on genes and gene products in the blood group systems.

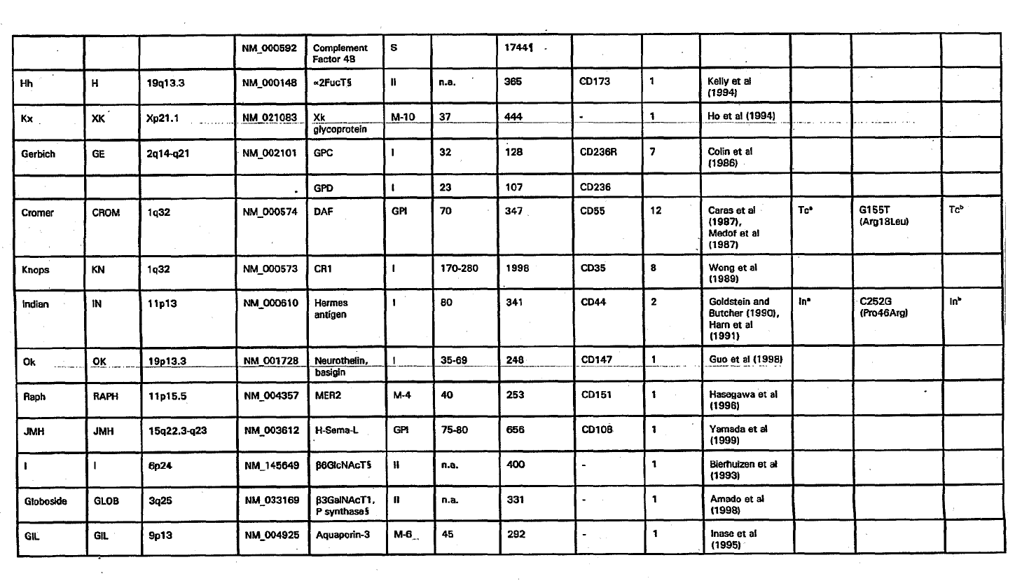

FIG. 2 shows a table of amino acid sequences of the Rhc peptide panel spanning the Leu60Ile, Asn68Ser and Pro103Ser polymorphisms determining the Rhc allele expression. The Rhc polymorphisms are indicated in bold.

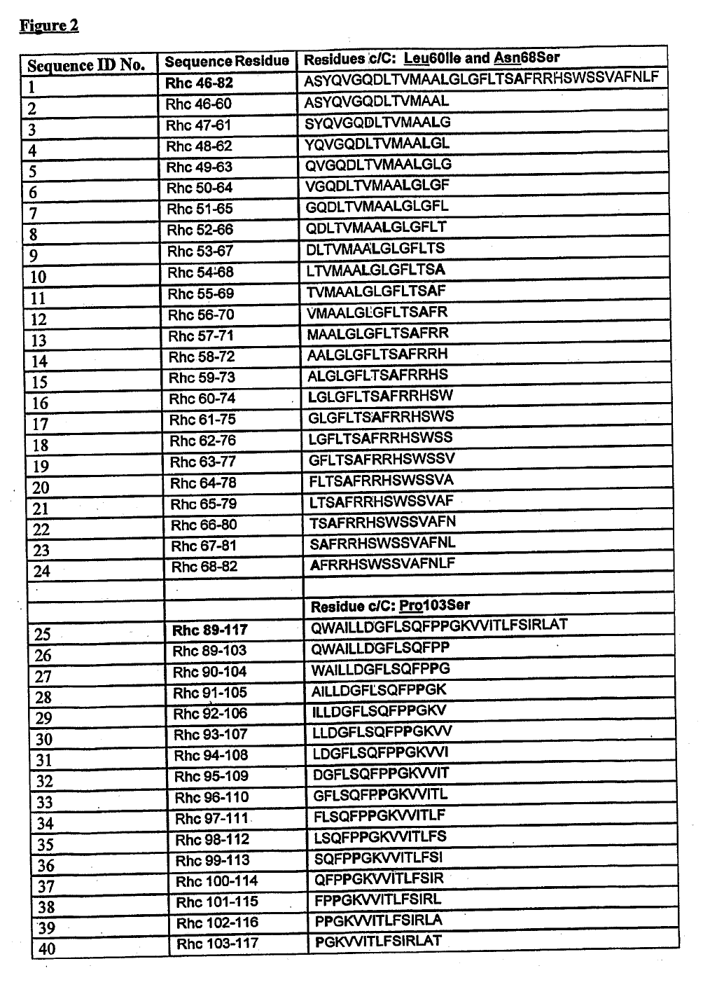

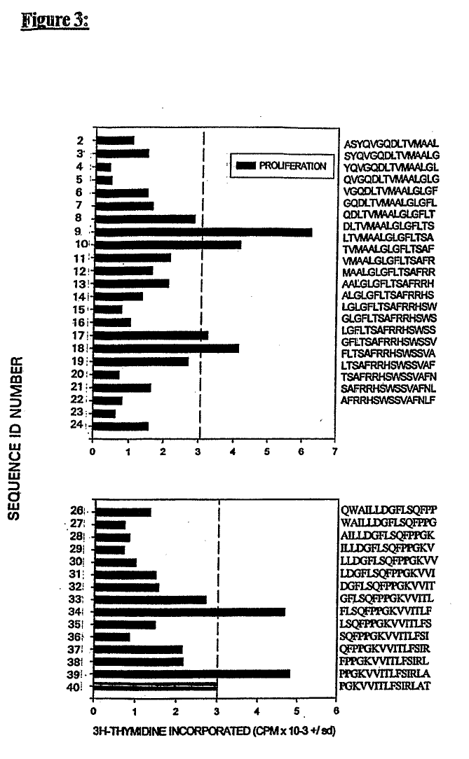

FIG. 3 shows T-cell proliferation responses to Rhc peptides in donor 1.

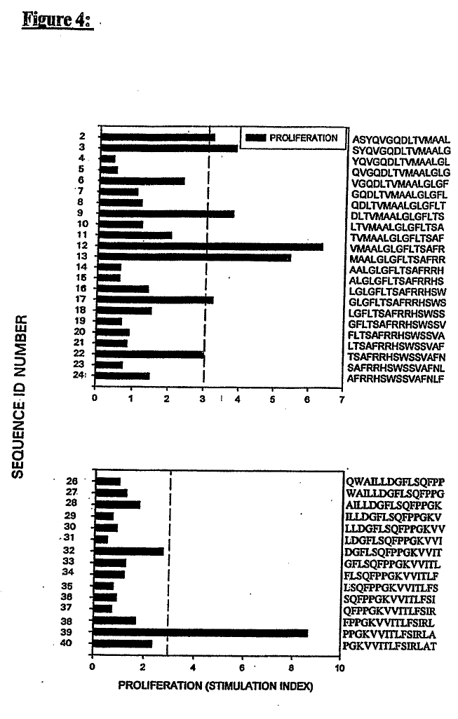

FIG. 4 shows T-cell proliferation responses to Rhc peptides in donor 2.

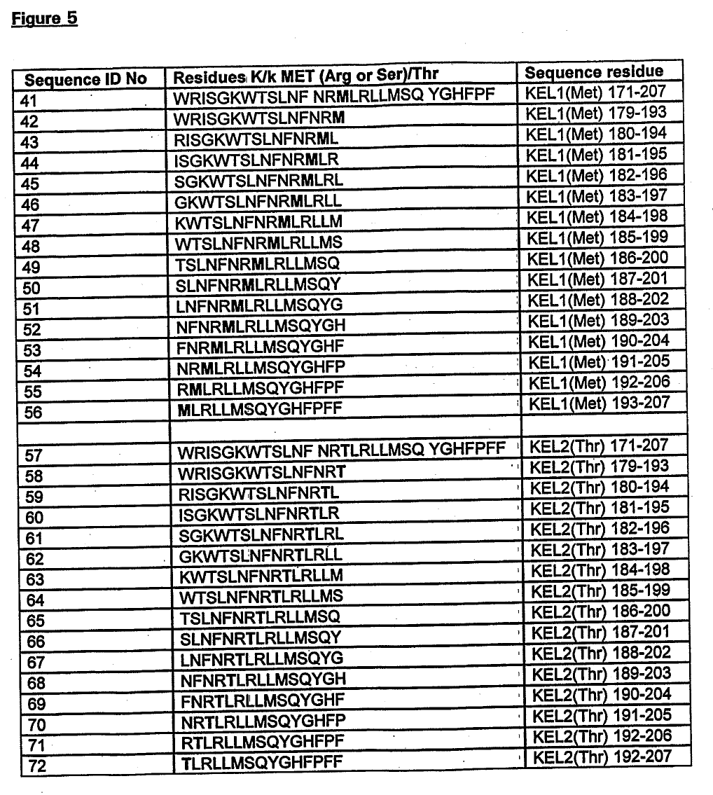

FIG. 5 shows a table of amino acid sequences of the Kell peptide panel spanning the 193 Met/Thr polymorphism determining the K/k allele expression. The K/k polymorphism is indicated in bold.

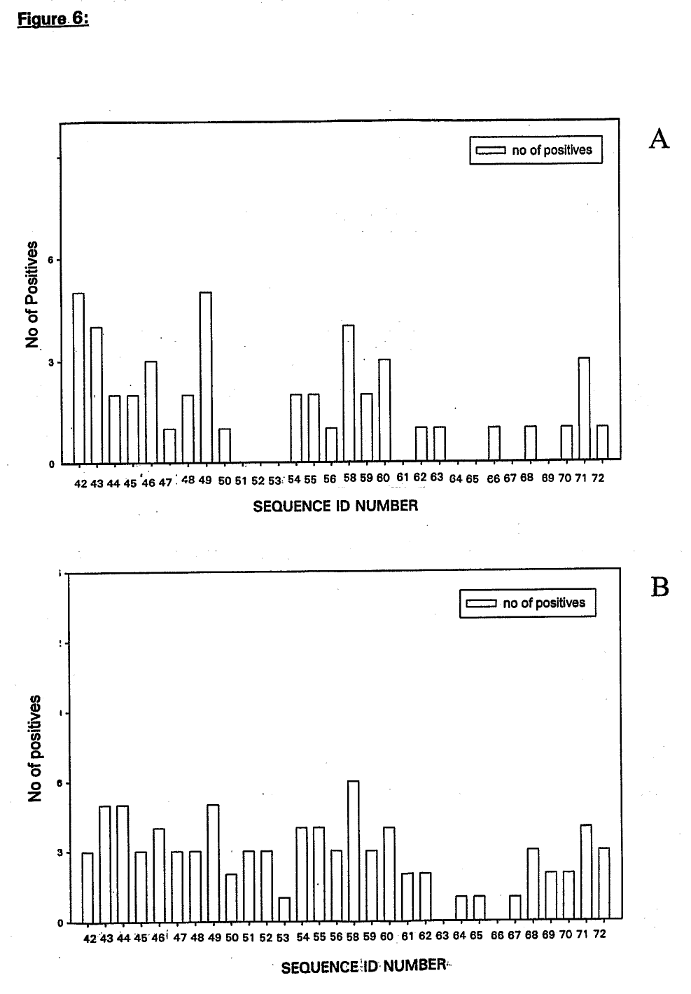

FIG. 6A shows T-cell proliferation responses to Kell peptide panel by K1 negative alloimmunised donors (n=6).

FIG. 6B shows T-cell proliferation responses to Kell peptide panel by K2 negative alloimmunised donors (n=6).

### EXAMPLE 1

### Rhc

The Rhc antigen resides on the Rh blood group protein, and is expressed on the RBC surface at a level of 20-30,000 copies per cell. The Rhc antigen is antithetical to the RhC antigen. Rhc differs from the RhC at 4 different positions: Trp16Cys, Leu60Ile, Asn68Ser and Pro103Ser. The Pro103 is thought to be the most important in the Rhc antigen expression.

Donor

Blood samples were obtained from healthy Rhc negative donors whose sera contained anti-Rhc alloantibodies. All the donors had anti-Rhc alloantibodies as a result of antigen mismatch during pregnancy or blood transfusion and not as a result of deliberate alloimmunisation. Control blood samples were obtained from healthy Rhc negative individuals whose sera contained no anti-Rhc alloantibodies. All blood samples were taken by venupuncture into citrate or preservative-free heparin anticoagulant.

Peptides and Mitogens

Peripheral blood mononuclear cells (PBMC) were stimulated with sets of overlapping 15-mer peptides, which included the Rhc polymorphisms (Leu60Ile, Asn68Ser and Pro103Ser) (Pepceuticals Ltd, BioCity, Nottingham) (FIG. 2). The Trp16Cys polymorphism was not tested, since this is common to the RhD antigen. The peptides were added to cultures at a concentration of 20 μg/ml. The control antigen Mycobacterium tuberculosis purified protein derivative (PPD) (Statens Seruminstitut, Denmark) was dialysed extensively against phosphate buffered saline pH 7.4 (PBS) and filter sterilized before addition to cultures at 10 μg/ml. PPD readily provokes recall T-cell responses since most UK citizens have been immunized with the bacille Calmette-Guérin vaccine. The mitogen concanavalin A (Con A) (Sigma, Poole, Dorset, UK) was used to stimulate cultures at 1 μg/ml.

Isolation of PBMC

PBMC were isolated from donor's blood using density gradient centrifugation (Lymphoprep; Nycomed, Denmark). The viability of PBMC was greater than 90% in all experiments as shown by staining cells with trypan blue.

Proliferation Assays

PBMC were cultured in 100 μl volumes in microtitre plates at a concentration of 1.25×10⁶ cells per ml in the α modification of Eagle medium (Gibco, Paisley, United Kingdom) supplemented with 5% autologous serum, 4 nmol/L L-glutamine (Sigma), 100 UmL sodium benzylpenicillin G (Sigma). All plates were incubated at 37° C. in a humidified atmosphere of 5% carbon dioxide and 95% air. The cell proliferation in cultures was estimated from the incorporation of ³H-thymidine in triplicate wells 5 days after stimulation with antigen as described previously (Barker et al 1997 Blood, 90; pages 2701-2715). Proliferation results are presented as a stimulation index (SI), expressing the ratio of mean CPM in stimulated versus unstimulated control cultures. An SI>3 are interpreted as representing a significant positive response.

Results

PBMC were obtained from healthy Rhc negative donors whose sera contained anti-Rhc alloantibodies and stimulated with the panels of peptides containing the Leu60Ile, Asn68Ser and Pro103Ser polymorphisms, corresponding to Rhc expression. The results from two donors are illustrated in FIGS. 3 and 4. It can be seen that particular peptides were stimulatory in each case, and that some sequences elicited proliferation in both individuals. For example, SEQ ID NOS 9, 17 or 39 stimulated proliferative responses by T-cells from both donors. It should also be noted that peptides containing each of the three polymorphisms were capable of being stimulatory.

Control experiments demonstrated that the observed T-cell responses are a consequence of exposure of Rhc-negative individuals to Rhc positive RBC. Thus, PBMC from Rhc-positive control donors did not respond to any peptides containing SEQ ID NOS 9, 17 or 39, and control peptides corresponding to the antithetical Rhc-negative sequences were not stimulatory in either donor group.

Conclusion

The epitope mapping technique is able to identify particular peptides from the Rhc sequence that are recognized by T-cells from Rhc-negative donors who have anti-Rhc alloantibodies. We conclude that these T-cells are responsible for providing help for the anti-Rhc alloantibody production. The peptides identified represent tolerogens, with the ability therapeutically to prevent or switch off responses to the Rhc blood group in Rhc-negative individuals. Tolerisation may be achieved by, for example, delivery to mucosal surfaces such the nasal passages.

### EXAMPLE 2

### KELL (K1)

The Kell blood group antigen system is a highly polymorphic system, consisting of over 25 different antigens, which are expressed on the Kell protein. The Kell protein is a type II membrane glycoprotein expressed on the surface of erythrocytes and has been found to be present on the surface of red cells at a level of 3500 to 17000 copies per cell. The most clinically and immunologically important of the Kell antigens is the Kell 1 (K1) antigen and its antithetical partner Kell 2 (K2). K1 differs from K2 by a single nucleotide polymorphism (SNP) at amino acid position 193 in the antigen sequence. This results in the expression of methionine at position 193 in the K1 antigen and threonine at this position in the K2 antigen (see FIG. 5).

Donors

Blood samples were obtained from 6 healthy K1 negative donors whose sera contained anti-K1 alloantibodies. All the donors had anti-K1 alloantibodies as a result of antigen mismatch during pregnancy or blood transfusion and not as a result of deliberate alloimmunisation. Control blood samples were obtained from 8 healthy K1 negative individuals whose sera contained no anti-K1 alloantibodies. All blood samples were taken by venupuncture into citrate or preservative-free heparin anticoagulant.

Peptides and Mitogens

Peripheral blood mononuclear cells (PBMC) were stimulated with two sets of overlapping 15-mer peptides, which correspond to the K1/K2 polymorphism (Pepceuticals Ltd, BioCity, Nottingham). One set of peptides represents the K1 polymorphism and expresses the methionine amino acid at every possible position along the peptide and the other set represents the K2 polymorphism, expressing threonine at every position along the peptide (FIG. 5). The peptides were added to cultures at a concentration of 20 μg/ml. The control antigen Mycobacterium tuberculosis purified protein derivative (PPD) (Statens Seruminstitut, Denmark) was dialysed extensively against phosphate buffered saline pH 7.4 (PBS) and filter sterilized before addition to cultures at 10 μg/ml. PPD readily provokes recall T-cell responses since most UK citizens have been immunized with the bacille Calmette-Guérin vaccine. The mitogen concanavalin A (Con A) (Sigma, Poole, Dorset, UK) was used to stimulate cultures at 10 μg/ml.

Isolation of PBMC

PBMC were isolated from donor's blood using density gradient centrifugation (Lymphoprep; Nycomed, Denmark). The viability of PBMC was greater than 90% in all experiments as shown by staining cells with trypan blue.

Proliferation Assays

PBMC were cultured in 100 μl volumes in microtitre plates at a concentration of 1.25×10⁶ cells per ml in the α modification of Eagle medium (Gibco, Paisley, United Kingdom) supplemented with 5% autologous serum, 4 nmol/L L-glutamine (Sigma), 100 UmL sodium benzylpenicillin G (Sigma). All plates were incubated at 37° C. in a humidified atmosphere of 5% Carbon dioxide and 95% air. The cell proliferation in cultures was estimated from the incorporation of ³H-thymidine in triplicate wells 5 days after stimulation with antigen as described previously (Barker et al 1997 Blood, 90; pages 2701-2715). Proliferation results are presented as a stimulation index (SI), expressing the ratio of mean CPM in stimulated versus unstimulated control cultures. An SI>3 are interpreted as representing a significant positive response.

Results

PBMC from all alloimmunised donors responded to one or more of the Kell peptides. From the data, however, it appears that donors' PBMC responded better to the K1(193Met) peptides than to the K2(193 Thr) set and in particular to those peptides with 193Met near the C-terminus (FIG. 6A). For example, SEQ ID NO 41 induced significant proliferation by PBMC from 83.3% of donors and SEQ ID NO 42 elicited proliferation by PBMC from 64% of donors. PBMC from donors also responded significantly to SEQ ID NO 48 from the K1(193Met) set of peptides, with 83.3% of donors responding significantly to this peptide. This suggests that there are immunodominant T-helper cell epitopes formed when these peptide sequences bind to the restricting MHC class II molecules, which are responsible for driving the T-helper cell response to the K1 antigen.

The results from alloantibody-negative control donors' PBMC revealed higher responses to the Kell peptide panel than seen in previous studies of responses against other blood groups by unimmunised individuals. Thus PBMC from each of the control donors responded to one or more of the Kell peptides (FIG. 6B). However, upon comparison with the results from the alloimmunised donors, the responses seen in the control group differ in not being focused on peptides with 193Met near the C-terminus, confirming the importance of this particular register for T helper cell recognition. We propose that the responses to the peptides containing the polymorphism in other registers are the result of cross-reaction with environmental antigens, such as bacterial proteins, by PBMC from the control donors, and therefore not a consequence of responses to Kell. A second possibility is that the high responses in the control group could be the result of a strong primary response by donors' PBMC in response to the Kell peptides.

Conclusion

The results from the alloimmunised donors have shown that PBMC from these donors respond mainly to the K1(193Met) peptides and in particular to those peptides with 193Met at the C-terminus. This leads us to conclude that there are immunodominant T-helper cell epitopes formed with the polymorphism in this register within the peptide sequences. Donors also showed significant proliferation to K1 SEQ ID NO 48, suggesting that expression of the polymorphism in the middle of the peptide sequence may also provide T-helper cells with a second epitope. PBMC from the control unimmunised donors show strong background proliferative responses to the Kell peptides, but, unlike the immune donors, these were not focused on the peptides with the polymorphism at the C-terminus or position 8.

Responses to peptides with 193Met at the C-terminus and at position 8 are associated with the alloantibody response to Kell, and we conclude that the T-cells providing help for alloantibody production recognise these sequences. The peptides identified represent tolerogens, with the ability therapeutically to prevent or switch off responses to the K1 blood group in K1-negative individuals. Tolerisation may be achieved by, for example, delivery to mucosal surfaces such the nasal passages.

1. A method of identifying an immunologically effective epitope, the method comprising:
a) preparing an oligomer of a protein present on the surface of a red blood cell which includes a polymorphism therein and b) ascertaining whether the oligomer stimulates T-cells.

2. The method according to claim 1 wherein the oligomer is between 10 and 20 nucleotides in length.

3. The method according to claim 2 wherein the oligomer is 15 nucleotides in length.

4. The method according to claim 1 wherein a set of oligomers is prepared.

5. The method according to claim 4 wherein the oligomers are overlapping.

6. The method according to claim 1 wherein the polymorphism is a single nucleotide polymorphism.

7. The method according to claim 1 wherein the polymorphism is unique to the protein.

8. The method according to claim 1 wherein the polymorphism is in the middle region of the oligomer.

9. The method according to claim 1 wherein the protein is selected from the group consisting of ABO, MNS, P, Rh, Lutheran, Kell, Lewis, Duffy, Kidd, Diego, Yt, Xg, Scianna, Dombrock, Colton, Landsteiner-Wiener, Chido-Rodgers, Hh, Kx, Gerbich, Cromer, Knops, Indian, Ok, Raph, JMH, I, Globoside or and GIL.

10-15. (canceled)

16. A method for the tolerisation of a subject that has been exposed to an antithetical allele, the method comprising administering to the subject a composition comprising an immunologically effective epitope identified by the method of claim 1.

17. A method for the tolerisation of a subject that has been exposed to an antithetical allele, the method comprising administering to the subject a composition comprising an immunologically effective non-rhesus surface antigen protein or a peptide fragment thereof.

18. The method according to claim 17 wherein the non-rhesus surface antigen protein is selected from the group consisting of ABO, MNS, P, Lutheran, Kell, Lewis, Duffy, Kidd, Diego, Yt, Xg, Scianna, Dombrock, Colton, Landsteiner-Wiener, Chido-Rodgers, Hh, Kx, Gerbich, Cromer, Knops, Indian, Ok, Raph, JMH, I, Globoside and GIL.

19. The method according to claim 17 wherein the exposure to the antithetical allele has resulted in a condition selected from: haemolytic disease of the fetus and newborn and haemolytic transfusion reaction.

20. The method according to claim 16 wherein the composition is formulated for delivery through mucosal tissue.

21. The method according to claim 17 wherein the composition is formulated for delivery through mucosal tissue.

22. The method according to claim 17 wherein the protein is Kell or the peptide fragment has a sequence selected from the group consisting of SEQ ID Nos: 42, 43, 46, 49, 58, 60 and 71.
